(19) United States
(12) Patent Application Publication
(10) Pub. No.: US 2023/0087601 A1
(43) Pub. Date: Mar. 23, 2023

(54) COSMETIC COMPOSITIONS CONTAINING LOW MOLECULAR WEIGHT AMORPHOUS GRADE POLYLACTIC ACID RESIN

(71) Applicant: NatureWorks LLC, Plymouth, MN (US)

(72) Inventors: William D. Coggio, Hudson, WI (US); John Mark Chandler, Bear, DE (US); Samantha Leigh Thompson-Barclay, Plymouth, MN (US)

(21) Appl. No.: 17/799,754

(22) PCT Filed: Feb. 19, 2021
(86) PCT No.: PCT/US2021/018658

§ 371 Date: Aug. 15, 2022

Related U.S. Application Data
(60) Provisional application No. 62/979,556, filed on Feb. 21, 2020.

(51) Int. Cl. A61K 8/85; A61Q 1/06; A61Q 1/02; A61Q 17/04; A61K 8/06; A61K 8/73; A61K 8/27; A61K 8/34; C08L 91/06; C08L 67/04

(52) U.S. Cl. CPC A61K 8/85; A61Q 1/06; A61Q 1/02; A61Q 17/04; A61K 8/062; A61K 8/064; A61K 8/73; A61K 8/27; A61K 8/342; C08L 91/06; C08L 67/04; A61K 2800/43; A61K 2800/48; C08L 2205/035

(57) ABSTRACT

Cosmetic compositions contain certain polylactic acid resins as film-formers. The polylactic acid resin is effective at low levels in the composition and is compatible with many organic ingredients commonly used in cosmetic compositions. The cosmetic composition can be formulated as a spray, a lotion, a cream or a paste.

This invention relates to cosmetic compositions.

Cosmetics are products that are applied to the skin to alter its appearance, perform a protective function, and/or to perform a healing or other medicinal function. These products are often formulated as emulsions that contain an aqueous phase and an oil phase. When the aqueous phase is continuous and the oil phase is discontinuous and dispersed within the aqueous phase, the emulsion is referred to as an oil-in-water emulsion. Conversely, emulsions in which the oil phase is continuous and a discontinuous aqueous phase is dispersed within the oil phase are known as water-in-oil emulsions.

Cosmetics are often formulated to contain “film-formers”, which are typically polymers that, upon application to the skin and removal of volatiles, produce a pliable and cohesive film on the skin. Among other functions, the film can serve as a matrix that holds active ingredients on or near the surface of the skin, where their function is needed. Film-formers often are used to reduce smudging and to help prevent transfer. A wide variety of polymeric materials have been proposed for use as film-formers for cosmetic products as illustrated, for example, in U.S. Pat. Nos. 6,342,209 and 6,726,900.

It has now been discovered the certain polylactic acid resins are useful film-formers in cosmetic products.

Therefore, in one aspect, this invention is a cosmetic composition, comprising (component 1) at least one of (i) a water-immiscible organic compound, having a molecular weight of up to 1000 g/mol, a melting temperature no greater than 60° C. and a boiling temperature at 101 kPa pressure of at least 100° C., and (ii) a water-immiscible silicone; and (component 2) an amorphous grade polylactic acid resin containing at least 70% by weight of lactic repeating units and having a glass transition temperature of no greater than 65° C., wherein the polylactic acid resin has a number-average molecular weight of 1000 to 12,000 g/mol and contains a residue after removal of hydroxyl groups of a hydroxyl-containing initiator compound having 2 to 24 carbon atoms and 1 to 8 hydroxyl groups, the polylactic acid resin constituting 0.1 to 10% of the total weight of the cosmetic composition and being dissolved in component 1 or an oil phase in which component 1 also is dissolved.

Polylactic acid resins having the enumerated characteristics have been found to perform well as film-formers in cosmetic products.

The term “polylactic acid resin” used herein to denote polymers having at least 70% by weight of polymerized lactic repeating units (i.e., repeating units having the structure —OC(O)CH(CH₃)—), irrespective of how those lactic units are formed into the polymer. Any lactic repeating unit may be of the L or D configuration. The polylactic acid resin may contain at least 75%, at least 80%, or at least 85% by weight of lactic units.

The polylactic acid resin may further contain repeating units derived from other monomers that are copolymerizable with lactide or lactic acid, such as alkylene oxides (including ethylene oxide, propylene oxide, butylene oxide, tetramethylene oxide, and the like), cyclic lactones (including caprolactone and the like), glycolide, hydroxyacids such as 3-hydroxybutyric acid and 3-hydroxyhexanoic acid, or carbonates (including ethylene carbonate and the like). Repeating units derived from these other monomers can be present in block, quasi-random, and/or random arrangements. These other repeating units suitably constitute up to 25%, up to 15%, up to 10%, up 5%, or up to 2% by weight of the polylactic acid, and may be absent.

The polylactic acid resin contains a residue of an initiator compound having 1 to 8 hydroxyl groups and 2 to 24 carbon atoms. An initiator compound is a molecule having at least one hydroxyl group which reacts with a molecule of lactic acid or lactide during the course of polymerization to initiate polymer growth, the residue after reaction of the initiator compound becoming incorporated into the polylactic acid molecule. The initiator after removal of hydroxyl groups preferably has a molecular weight of up to 400 g/mol.

Among the useful initiator compounds are monoalcohols which preferably are aliphatic. Such monoalcohols may be linear, branched, and/or cyclic, saturated or unsaturated, and more preferably contain 6 to 20 carbon atoms. Examples include 1-propanol, 2-propanol, 1-butanol, 1-pentanol, 1-hexanol, 2-ethyl-1-butanol, 2-ethyl-1-hexanol, n-octanol, n-decanol, 2-ethyl-1-octanol, n-dodecanol, 2-ethyl-1-decanol, n-tetradecanol, 2-ethyl-1-dodecanol, n-hexadecanol, 2-ethyl-1-tetradecanol, 1-octadecanol, 2-ethyl-1-hexadecanol, various Guerbet alcohols not specifically mentioned above, cis-9-octadecen-1-ol, 1-icosonol, and 1-docosonol. Those having at least 6 or at least 8 carbon atoms and up to 18 carbon atoms sometimes offer particular advantages.

Among the polyhydroxy compounds that are useful as initiators are ethylene glycol, propylene glycol, diethylene glycol, dipropylene glycol, triethylene glycol, tripropylene glycol, glycerine, trimethylolpropane, pentaerythritol, 1,4-butanediol, 1,6-hexanediol, 1,6-octanediol, polyether polyols having 10 to 24 carbon atoms and a molecular weight of up to 400 g/mol, and the like. In some embodiments, the polyhydroxy compound is a linear or branched aliphatic polyol having 3 to 9 carbon atoms and 2 to 4 hydroxyl groups.

In some embodiments, the initiator contains no carboxyl (—COOH) or carboxylate (—COO⁻) groups.

The initiator residue preferably constitutes no more than 30%, no more than 20%, or no more than 12% of the weight of the polylactic acid resin.

The polylactic acid resin is an amorphous grade. For purposes of this invention, a polylactic acid resin is considered to be an amorphous grade if, as a neat material (i.e., uncombined with any other material) it forms no more than 5 J/g of PLA crystallites when quiescently (i.e., under no applied strain) heated at any temperature in the range of 70° to 140° C. for one hour. Crystallinity is measured on a sample of the polylactic acid resin which has been previously heated to at least 220° C. to melt any crystallites and then quenched by rapidly cooling to room temperature (23±3° C.). The quenched sample is then heated to a temperature within the range of 70° to 140° C., kept isothermally for one hour, and again quenched by cooling to room temperature. Crystallinity then is conveniently measured using differential scanning calorimetry (DSC) methods. The amount of such crystallinity is expressed herein in terms of J/g, i.e., the enthalpy of melting, in Joules, of the polylactic acid crystals in the sample, divided by the weight in grams of polylactic acid(s) in the sample. A convenient test protocol for making DSC measurements is to heat a 5-10 milligram sample from 25° C. to 225° C. at 20° C./minute under air, on a Mettler Toledo DSC 821e calorimeter running Star V. 6.0 software, or equivalent apparatus.

The number-average molecular weight of the polylactic acid resin is 1000 g/mol to 12,000 g/mol, as measured by gel permeation chromatography against linear polystyrene standards. A preferred minimum number-average molecular weight is at least 2000 g/mol and a preferred maximum number average molecular weight is up to 10,000 g/mol, up to 8000 g/mol, or up to 6000 g/mol.

The polylactic acid resin may have, as a neat material, a glass transition temperature of no greater than 65° C., no greater than 55° C., no greater than 50° C., or no greater than 40° C., as measured by differential scanning calorimetry at a temperature ramping rate of 20° C./minute, the glass transition temperature being taking as the midpoint of the transition slope.

The lactic units in the polylactic acid resin may be either the L- or D-enantiomer, or a mixture thereof. L- and D-lactic units, when both are present, may be distributed regularly, randomly, or pseudo-randomly in the polylactic acid resin molecules.

In some embodiments, the polylactic acid resin is made by polymerizing lactide. Such a polylactic acid resin may be a polymer of L-lactide, D-lactide, rac-lactide, meso-lactide, or a mixture of any two or more thereof. The polylactic acid resin may be a polymer of a monomer mixture containing 75% to 100% meso-lactide and 0% to 25% of another lactide (L-lactide, D-lactide, and/or rac-lactide). In certain embodiments, the polylactic acid resin contains 20% to 80%, preferably 40% to 60%, of one lactic acid enantiomer (i.e., either L- or D-lactic units) and conversely 80% to 20%, preferably 60% to 40%, of the other lactic acid enantiomer, based on the total weight of lactic units.

Alternatively, the polylactic acid resin may be made by other methods, such as the direct polymerization of lactic acid, a lactic acid salt, or an alkyl ester of lactic acid; by a transesterification process in which an oligomeric lactic acid is reacted with an initiator compound; or by various solid state polymerization methods.

The polylactic acid resin in certain embodiments is represented by the structure:

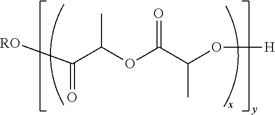

wherein y is 1 to 8, especially 1 to 6 or 1 to 4, and x is a positive number such that the polylactic acid resin has a molecular weight as described before. R is the residue after removal of hydroxyl groups of a hydroxyl-containing initiator compound as described before. R preferably contains 2 to 24 carbon atoms and preferably lacks carboxyl or carboxylate groups. When y is greater than one, R preferably contains 3 to 9 carbon atoms. When y is one, R preferably contains 6 to 20, 6 to 18 or 8 to 18 carbon atoms.

The polylactic acid resin may, for example, constitute 0.25% to 10% by weight of the cosmetic composition. In some embodiments, the polylactic acid resin constitutes up to 8%, up to 6%, up to 5%, up to 4%, or up to 3% of the weight of the cosmetic composition, and at least 0.5% by weight thereof.

The cosmetic composition contains (component 1) at least one of (i) a water-immiscible organic compound, having a molecular weight of up to 1000 g/mol, a melting temperature no greater than 50° C., and a boiling temperature at 101 kPa pressure of at least 100° C.; and (ii) a water-immiscible silicone compound. The polylactic acid resin is dissolved in component 1 or dissolved in an oil phase in which component 1 is also dissolved.

Component 1 compounds may constitute at least 2%, at least 5%, at least 10%, or at least 20% of the total weight of the cosmetic composition, and may constitute up to 99%, up to 90%, up to 70%, up to 60%, or up to 50% thereof.

A material is considered as “water-immiscible” herein if it is soluble in water to the extent of no more than 1 part by weight per 100 parts by weight water at 25° C.

Examples of such water-immiscible organic compounds include (a) nonionic organic compounds having a molecular weight of at least 190 g/mol and up to 1000 g/mol, at least one amido or ester group, and an alkyl chain containing at least 8 carbon atoms. Water-immiscible organic compounds (a) include various fatty acid esters, fatty acid amides and esters, or esters of fatty alcohols, each of which can be naturally-occurring (such as an animal or plant oil) or synthetic. Specific examples include triglycerides such as triheptanoin, various animal fat and plant oils such as coconut oil, shea butter, olive oil, argan oil, canola oil, palm oil, meadowfoam seed oil and avocado oil, and capric/caprylic triglyceride; mono- or diacetylated mono- or di-fatty acid glycerides prepared from a plant or vegetable oil feedstock; C₃₋₂₄ (especially C₆₋₂₄) dialkyl esters of dicarboxylic acids, such as di(octenyl)succinate, di(ethylhexyl)naphthanate, di(ethylhexyl)phthalate, diisopropyl sebacate and di-n-butyl sebacate; and C₆₋₂₄ alkyl esters of C₆₋₂₄ fatty acids, such as ethylhexylpalmitate.

Other examples of such water-immiscible organic compounds include (b) C₈₋₅₀ alkanes or mixtures of such alkanes, including various mineral oils and paraffins, which alkanes or mixtures of alkanes may be liquid or solid at 23° C., (c) fatty alcohols having 8 or more carbon atoms, such as 8 to 50 or 8 to 24 carbon atoms, (d) waxes not included within any of (a) to (c), including for example, natural waxes such as beeswax, rice bran wax, bayberry wax, lanolin, carnauba wax, candelilla wax, ouricury wax, sugarcane wax, jojoba wax, epicuticular wax, ozokerite wax, and coconut wax as well as synthetic waxes such as paraffin wax and microcrystalline wax, (e) water-immiscible organic UV absorbers including homosalate, octisalate, octocrylene, octinoxate, avobenzone, octylmethoxyl cinnamate, and ethylhexyl salicylate, and (f) water-immiscible C₃₋₂₄ dialkyl esters of C₂₋₇ linear aliphatic dicarboxylic acids, such as diisopropyl adipate, di-n-butyl adipate, diisopropyl succinate and di-n-butyl succinate.

Water-immiscible silicone compounds include nonionic organosilicones such as hexamethyldisiloxane, octamethyltrisiloxane, decamethyltetrasiloxane octamethylcyclotetrasiloxane, decamethylcyclopentasiloxane, and linear or branched polydimethylsiloxane (PDMS) oil such as hexamethyldisiloxane, octamethyltrisiloxane, decamethyltetrasiloxane, dodecamethylpentasiloxane, polymethylhydrosiloxane (PMHS) oil, and other liquid cyclomethicones.

The cosmetic composition may contain one or more surfactants (emulsifiers or wetting agents), particularly when an aqueous phase is present. Such surfactants may be present, for example, to prevent separation of the oil and aqueous phases and/or to wet out the surface of pigments or other particulate solids so they can be dispersed into the composition. A surfactant may be soluble in component (1), may be soluble or self-dispersible in water, or both. The surfactant may be nonionic, anionic, cationic, or zwitterionic, or a mixture of any two or more thereof. Examples of useful surfactants include sorbitan esters such as sorbitan monooleate, sorbitan stearate, sorbitan laurate, sorbitan sesquioleate, sorbitan tristearate, sorbitan palmitate, and sorbitan trioleate; ethoxylated and esterified sorbitans such as polysorbate 20, polysorbate 60, and polysorbate 80; ethoxylates of fatty acids; propylene oxide/ethylene oxide block copolymers; ethoxylated amines; ethoxylated fatty acid amides, poloxamers, mono- and di-fatty acid esters of glycerol such as glycerol monostearate, glycerol monooleate and glycerol monolaurate; mono-fatty acid esters of a C₂-C₆ polyol such as propylene glycol, ethylene glycol, diethylene glycol, dipropylene glycol, 1,4-butane diol, pentaerythritol, trimethylolpropane, trimethylolethane; alkylphenol ethoxylates; dibasic esters of an aliphatic (preferably linear C₄₋₁₂) dicarboxylic acid and an alkylene ether or polyether monol (an example of such a dibasic ester being di(ethoxyethyl) succinate); alkyl polyglucosides; amine oxides; diacylated fatty acid monoglycerides; and the like. The surfactant(s), when present, may constitute 1% to 40% of the total weight of the cosmetic composition. A preferred amount is 1% to 20% or 2% to 15%, on the same basis.

In some embodiments, the cosmetic composition includes at least one nonionic surfactant that is soluble in one or more water-immiscible compound(s) present in the cosmetic composition. Such a surfactant may have a hydrophilic-lipophilic balance of up to 12, preferably up to 10. When an aqueous phase is present, the cosmetic composition may include at least one additional nonionic surfactant that is soluble or self-dispersible in water and has a hydrophilic-lipophilic balance of at least 10, preferably at least 12. A cosmetic composition of the invention may include at least one nonionic surfactant having a hydrophilic-lipophilic balance of up to 12 and which is soluble in one or more water-immiscible compound(s) present in the cosmetic composition and at least one different nonionic surfactant having a hydrophilic-lipophilic balance of at least 10, preferably at least 12, and which is soluble or self-dispersible in water.

The cosmetic composition may contain an aqueous phase. The aqueous phase includes water and optionally one or more water-soluble ingredients dissolved in the water. The aqueous phase, when present, may constitute 0.5% to 95% of the total weight of the cosmetic composition. In particular embodiments, the aqueous phase may constitute at least 1%, at least 5%, at least 10%, or at least 20% of the total weight of the cosmetic composition, and may constitute, for example, up to 80%, up to 60%, or up to 50% thereof. The weight of the aqueous phase includes that of any surfactants that are soluble in water or are self-dispersible in water. Water by itself may constitute, for example, 5% to 80%, 5% to 60%, or 5% to 50% of the total weight of the cosmetic composition.

When an aqueous phase is present, the cosmetic composition may take the form of an oil-in-water emulsion or a water-in-oil emulsion. The oil phase includes component 1 as defined above and the polylactic acid resin. Such an emulsion may have particulate material dispersed in either or both of the oil and aqueous phases. Either the water or the oil phase may be predominant in terms of weight. The physical form of the product may be, for example, that of a low viscosity, sprayable liquid, a cream, a lotion, or a paste.

The aqueous phase, when present, may contain one or more water-soluble ingredients such as, for example, one or more humectants, one or more water-soluble polymers; one or more water-miscible solvents, one or more pH adjusters, and one or more water-soluble sunscreens. These water-soluble ingredients typically are dissolved in the water present in the cosmetic composition.

Among the useful humectants are alkylene diols and polyalkylene glycols, especially those having up to 8 carbon atoms and molecular weight of up to 130 g/mol, such as propanediol, 1,4-butanediol, 1,5-pentanediol, 1,2-pentanediol, 1,6-hexanediol, 1,2-hexanediol, and 1,8-octanediol. Other useful humectants include glycerine, glyceryl triacetate, and α-hydroxy acids having up to 10 carbon atoms, such as lactic acid, glycolic acid, 2-hydroxydecanoic acid, 2-hydroxyoctanoic acid, and oligomers thereof having up to 10 carbon atoms. Humectant(s), when present, may constitute 1% to 40% of the total weight of the cosmetic composition. A preferred amount is 2% to 30% or 5 to 25% on the same basis.

Water-soluble polymers may function as thickeners, dispersants, emollients, and/or perform one or more other functions. Examples of water-soluble polymers include styrene-maleic anhydride resins or esters thereof, in each case having weight average molecular weights of up to 7000 g/mol; water-soluble cellulose ethers; guar gum, maltodextrin, xanthan gum, hydroxypropyl starch phosphate, styrene-acrylic acid copolymer; ethylene-acrylic acid copolymer, polyacrylic acid, poly(ethylene glycol), poly(vinyl pyrrolidone) and poly(vinyl alcohol), pectin, dextrin, chitin, and carrageenan. Water-soluble polymer(s), when present, may constitute for example 0.05% to 5% of the total weight of the cosmetic composition, a preferred amount being 0.1% to 2% on the same basis.

A cosmetic composition of the invention may contain one or more organic solvents, which solvents may or may not be miscible with water. Examples of such solvents include compounds having zero or one hydroxyl groups, no carboxyl or carboxylate groups, and a molecular weight of up to 150 g/mol. Water-immiscible solvents include, for example, alkanes having up to 6 carbon atoms, benzene, toluene, and xylene. Water-miscible solvents include ethanol, methanol, 1-propanol, 2-propanol, t-butyl alcohol, pyridine, acetone, methyl ethyl ketone, diethyl ether, glyme, and diglyme. If present at all, these may constitute up to 50%, preferably up to 20%, up to 10%, up to 5%, or up to 2%, of the total weight of the cosmetic composition, and may be absent. When present, organic solvents often function as carriers for one or more other ingredients, and/or to reduce the viscosity of the cosmetic composition as a whole, or of an oil phase or aqueous phase of the cosmetic composition.

pH adjusters include amino acids such as arginine as well as various organic and inorganic acids and bases such as sodium hydroxide, potassium hydroxide, hydrochloric acid, phosphoric acid, monosodium phosphate, disodium phosphate, ammonium hydroxide, sodium carbonate, sodium hydrogen carbonate, and the like.

Phenylbenzimidazole sulfonic acid (ensulizole) is a useful water-soluble sunscreen.

The cosmetic composition may contain one or more pigments. For purposes of this invention, a pigment is a particulate solid having a melting or decomposition (if it does not melt) temperature in excess of 100° C. and which is not soluble in the remaining ingredients of the cosmetic composition. The pigment may be inorganic, such as titanium dioxide, iron oxide red, iron oxide yellow, iron oxide black, zinc oxide, carbon black, barium metaborate, calcium metaborate, chrome yellow, chromium oxide green, cuprous oxide, milori blue, molybdate orange, ultramarine blue, zinc chromate, zinc phosphate, activated carbon, graphite and the like, or may be an organic pigment such as toluidine red, bon maroon, red lake C, naphtol red, DNA orange, hansa yellow, diarylide yellow, phthalo blue, or phthalo green. Certain pigments may perform additional functions in the cosmetic composition when the composition is applied. Titanium dioxide and zinc oxide, for example, are UV blockers and are useful as such in sunscreen products. Such pigments may constitute, for example, 0.1% to 60% of the total weight of the cosmetic composition. A preferred amount is 1% to 25% or 1% to 15%. Sunscreen products may contain 5% to 60% by weight of titanium dioxide and/or zinc oxide.

The cosmetic composition may further contain one or more optional functional ingredients as is needed for the particular use for which it is formulated. These can be formulated into either the oil phase or the aqueous phase (when present) or both. Examples of useful functional ingredients include preservatives, electrolytes, perfumes, or other fragrances, perfume solubilizing agents, sequestering agents, emollients, protein hydrolysates and other protein derivatives, anti-aging or anti-wrinkle formulations, acne treatments, skin whiteners, medicinal agents, and propellants. In some cases, an ingredient may perform two or more functions in the cosmetic composition.

Specific of such functional ingredients include cucurbit[6]uril and bambus[6]uril; salicylic acid, coenzyme Q10, benzoate-4-methylbenzylidene, cinoxate, caprylhydroxamic acid, dioxybenzone, menthyl anthranilate, mexoryl SX, drometrizole, octocrylene, ethylhexyl salicylate, padimate 0, p-aminobenzoic acid (PABA), phenylbenzimidazole sulfonic acid, sulisobenzone, titanium trolamine salicylate, salicylic acid, retinoic acid (including the alltrans isomer known as tretinoin), benzoyl peroxide, hydroquinon, arbutun (including plant extracts containing same), kojic acid, azelaic acid, glycyrrhetic acid, levulinic acid, 2-cyano-3,3-diphenylacrylic acid, sodium benzotriazolyl butylphenol sulfonate, ethyl-2-cyan-3,3-diphenylacrylate, 2-t-butyl-6-(5-chloro-2H-benzotriazol-2-yl)-p-cresol, 2-(2-H-benzotriazol-2-yl)-4-methylphenol, benzophenone-12, bornelone, or 2-benzotriazolyl-4-tert-octylphenol, dihydroxyacetone, erythrulose, dihydroxyacetone-ortho-ethyl-acetate, canthaxanthin, afamelanotide, minoxidil, an 5-alpha reductase inhibitor such as dutasteride or ketoconazole, zinc pyrithione, selenium sulfide, clotrimazole, tea tree oil, or piroctone olamine, an amphetamine, an antihistamine, methylphenidate, oxymetazoline, tetrahydrolzoline hydrochloride, psilocybin, tea tree oil, piroctone olamine, chlorhexidine, octenidine, triclosan, sodium 3,5-dibromo-4-hydroxybenzenesulfonate (Dibromol), calcium thioglycolate, sodium thioglycolate, thioglycolic acid, ammonium thioglycolate, butyl thioglycolate, ethanolamine thioglycolate, glyceryl thioglycolate, isooctyl thioglycolate, isopropyl thioglycolate, magnesium thioglycolate, methyl thioglycolate, potassium thioglycolate, aluminum zirconium tetrachlorohydrex gly, aluminum chlorohydrate, aluminum chloride, resorcinol (“resorcin”), 1-napthol, p-aminophenol, p-phenylenediamine (and its salts), 4- amino-2- hydroxytoluene, phenoxyethanol, N, N-diethyl-m-toluamide, p-menthane-3,8- diol (active agent in the essential oil of lemon eucalyptus), nepetalactone (catnip oil), citronella oil, permethrin, neem oil, or bog myrtle extract.

Examples of useful fragrances include essential oils derived from berries, allspice, juniper, seeds, almond, anise, celery, cumin, nutmeg oil, bark, cassia, cinnamon, sassafras, wood, camphor, cedar, rosewood, sandalwood, agarwood, rhizome, galangal, ginger, leaves, basil, bay leaf, cinnamon, common sage, eucalyptus, lemon grass, melaleuca, oregano, patchouli, peppermint, pine, rosemary, spearmint, tea tree, thyme, wintergreen, resin, frankincense, myrrh, flowers, cannabis, chamomile, clary sage, clove, scented geranium, hops, hyssop, jasmine, lavender, manuka, marjoram, rose, rosemary, basil, lemon grass, ylang-ylang, peel, bergamot, grapefruit, lemon, lime, orange, tangerine, root, valerian, and mango.

Examples of other fragrances include furaneol, 1-hexanol, cis-3-hexen-1-ol, menthol, hexanal, cis-3-hexenal, furfural, fructone, hexyl acetate, ethyl methylphenylglycidate, methyl formate, methyl acetate, methyl butyrate, methyl butanoate, ethyl acetate, ethyl butyrate, ethyl butanoate, isoamyl acetate, pentyl butyrate, pentyl butanoate, pentyl pentanoate, benzoin, black pepper, cajuput oil, caraway, carrot seed, coriander, cypress, dill, fennel, helichyrsum, lavandin, lemon verena, bee balm, niaouli, palmarosa, petitgrain, tagetes, vetiver, dihydrojasmone, oct-1-en-3-one, 2-acetyl-1-pyrroline, 6-acetyl-2,3,4,5-tetrahydropyridine, γ-decalactone, γ-nonalactone, δ-octalactone, massoia lactone, sotolon, ethanethiol, grapefruit mercaptan, methanethiol, 2-methyl-2-propanethiol, myrcene, geraniol, nerol, citral, lemonal, geranial, neral citronellal, citronellol, linalool, nerolidol, limonene, camphor, terpineol, ionone, thujuon, benzaldehyde, eugenol, cinnamaldehyde, ethyl maltol, anisole, anethole, estragole, thymol, thiethylamine, trimethylamine, cadaverine, pyridine, indole, skatole, geraniol, geranyl acetate, linalool, linalyl acetate, tetrahydrolinalool, citronellol, citronellyl acetate, dihydromyrcenol, dihydromyrcenyl acetate, tetrahydromyrcenol, terpineol, terpinyl acetate, nopol, nopyl acetate, 2-phenylethanol, 2-phenylethyl acetate, benzyl alcohol, benzyl acetate, benzyl salicylate, benzyl benzoate, styrallyl acetate, amyl salicylate, dimethylbenzyl carbinol, trichloromethylphenylcarbinyl acetate, p-tert-butyl-cyclohexyl acetate, isononyl acetate, vetiveryl acetate, vetiverol, alpha-n-amylcinammic aldehyde, alpha-hexylcinammic aldehyde, 2-methyl-3-(p-tert-butylphenyl)-propanol, 2-methyl-3- (p-isopropylphenyl)-prop anal, 3-(tert-butylphenyl)-prop anal, tricyclodecenyl acetate, tricyclodecenyl propionate, 4-(4-hydroxy-4-methylpentyl)-3- cyclohexene carbaldehyde, 4-(4-methyl-3-pentenyl)-3-cyclohexene carbaldehyde, 4-acetoxy-3-pentyletetrahydropyran, methyl-dihydrojasmonate, 2-n-heptylcyclopentanone, 3-methyl-2-pentylcyclopentanone, n-decanal, 9-decenol-1, phenoxyethyl isobutyrate, phenylacetaldehyde dimethyl acetal, phenylacetaldehyde diethyl acetal, geranonitrile, citronellonitrile, cedryl acetate, 3-isocamphycyclohexanol, cedryl methyl ether, isolongifolanone, aubepine nitrile, aubepine, heliotropine, coumarin, eugenol, vanillin, diphenyl oxide, hydroxycitronellal, ionones, methylionones, isomethylioniones, irones, cis-3-hexenol and esters thereof, indane musk, tetralin musk, isochroman musk, macrocyclic ketones, macrolactone musk, ethylene brassylate, aromatic nitro-musk, bergamot oil, coriander oil, dimethyl heptanol, dimethyl benzyl carbinyl acetate, geranyl acetate, citronellyl acetate, rose synthetic, geranium bourbon, hedione, iso eugenol, methyl eugenol styrallyl acetate, stemone, rose oxide laevo, aldehyde C-II undecyclic, derivatives of 2,6-dimethyl-2-alkoxy octan-7-ol, vertivert oil, vetiverol, vetiveryl, acetate, quaiac wood oil, esters ol-anthranilic acid, benzyl salicylate, benzyl benzoate, oak moss, eugenol, p-tert-butyl cyclohexyl acetate and coumarin.

A cosmetic composition of the invention may be formulated as, for example, a sunscreen, a foundation, a concealer, a lipstick, a lip gloss, lip balm, mascara, an eyeliner, a blush, a bronzer, a body lotion, a primer, a highlight, a hairspray, an anti-wrinkle cream or lotion, or a face powder. Such a cosmetic composition may take the physical form of a solid (such as a stick, a bar, a powder, etc.), a liquid, a paste, a cream, or an ointment, as appropriate for its intended use. The oil phase and aqueous phase (when present) each may be liquids or solids at 23° C., although it is preferred that at least one of the phases (more preferably both) is a liquid or paste at that temperature. Either or both of the aqueous and oil phases may contain particles of a solid material such as a pigment, which solid material is insoluble in each of the phases.

The cosmetic composition is conveniently made by dissolving the polylactic acid resin in one or more of the water-immiscible compounds to produce an oil phase. This is conveniently performed at a temperature of, for example, 40° C. to 100° C. to melt or soften the materials, thereby facilitating mixing and dissolution. One or more surfactants may be combined into the oil phase. The oil phase is then combined with the remaining ingredients (if any) in one or more additional mixing steps. These mixing steps can be performed at a temperature of, for example, 10° C. to 100° C., as convenient.

The polylactic acid resin by itself tends to be somewhat viscous and it is generally convenient to heat it to 90° C. or above to facilitate combining it with the other materials. Such temperatures are sometimes inconveniently high for cosmetic manufacturing, it being generally preferred to restrict temperatures to 80° C. or below. For that reason, it is often convenient to provide the polylactic acid resin in the form of a premix with one or more other ingredients, especially a component 1 material and/or a surfactant that is soluble in a component 1 material. Such a premix may further contain one or more solvents and/or humectants (as described above) but preferably contains no more than 5% or no more than 2% by weight of water (if any at all). The premix then can be combined with other ingredients of the cosmetic formulation, in particular an aqueous phase, to produce a cosmetic.

A premix can be prepared by heat-softening the polylactic acid resin and combining the heat-softened polylactic acid resin with the other premix ingredients. The temperature is high enough to form a partially or entirely homogeneous mixture; 70 to 100° C. is suitable. The premix upon cooling is typically a wax or oily solid or semi-solid. Some phase separation may take place upon cooling to room temperature. An advantage of preparing such a premix is that it can be combined easily with other ingredients of a cosmetic formulation at somewhat reduced temperatures, compared to combining the polylactic acid with those other ingredients directly, due to its lower viscosity compared to that of the polylactic acid resin by itself. This allows lower temperatures to be used when the cosmetic is formulated, while still obtaining a good and highly uniform product. Using a premix therefore permits lower temperatures to be used when formulating the cosmetic, which can lead to further advantages such as less discoloration, less development of unwanted odors, and better product stability, especially when using natural ingredients that may be heat-sensitive.

Such a premix may contain, for example, at least 1%, preferably at least 5% or at least 10% by weight of the polylactic acid resin, and up to 50%, up to 40% or up to 30% thereof.

In some embodiments, such a premix includes at least one mono-fatty acid ester of a C₂-C₆ polyol such as propylene glycol, ethylene glycol, diethylene glycol, dipropylene glycol, 1,4-butane diol, pentaerythritol, trimethylolpropane, trimethylolethane. The fatty acid group of such an mono-fatty ester may contain, for example, 6 to 24 or 8 to 18 carbon atoms and may be saturated or unsaturated. Specific examples of fatty acid groups include stearate, oleate and laurate. Propylene glycol monooleate, propylene glycol monostearate, propylene glycol monolaurate, glycerol monostearate, glycerol monooleate and glycerol monolaurate are useful as the mono-fatty acid ester. Such a mono-fatty acid ester, when present, may constitute, for example, at least 25% or at least 40% or at least 50% of the total weight of the premix, and may constitute, for example, as much as 80% or as much as 75% of the total weight thereof.

In some embodiments, such a premix includes a component 1 material as described before. When present, such a component 1 material any constitute, for example, at least 1%, at least 2%, at least 5% or at least 10% of the total weight of the premix, and up to 60% or up to 50% thereof.

In some embodiments, such a premix contains a humectant or solvent as described before. When present, such a solvent or humectant may constitute, for example at least 1%, at least 5% or at least 10% of the total weight of the premix, and may constitute up to 50% thereof or up to 33% thereof.

In specific embodiments, such a premix contains (1) 1 to 50% by weight of the polylactic acid resin, (2) 25 to 75% by weight of at least one mono-fatty acid ester of a C₂-C₆ polyol such as propylene glycol, ethylene glycol, diethylene glycol, dipropylene glycol, 1,4-butane diol, pentaerythritol, trimethylolpropane, trimethylolethane, and at least one component 1 material, preferably at least one triglyceride and/or at least one C₃₋₂₄ dialkyl ester of an aliphatic (preferably linear aliphatic having up to 12 carbon atoms) dicarboxylic acid (and especially at least one plant oil or animal fat), and (3) 1 to 50% by weight of at least one component 1 material, especially a plant oil or animal fat, wherein (1), (2) and (3) together constitute at least 80%, at least 90% or at least 95% and up to 100% of the total weight of the premix. Such a premix may contain, for example, up to 20%, up to 10%, or up to 5% by weight of a surfactant, different from (2).

In other specific embodiments, such a premix contains (1) 1 to 50% by weight of the polylactic acid resin, (2) 5 to 50% of at least one solvent or humectant, and (3) 1 to 50% by weight of at least one component 1 material, preferably at least one triglyceride and/or at least one C₃₋₂₄ dialkyl ester of an aliphatic (preferably linear aliphatic having up to 12 carbon atoms) dicarboxylic acid (and especially at least one plant oil or animal fat), wherein (1), (2) and (3) together constitute at least 80%, at least 90%, or at least 95% and up to 100% of the total weight of the premix. Such a premix may contain, for example, up to 20%, up to 10%, or up to 5% by weight of a surfactant.

In other specific embodiments, such a premix contains (1) 1 to 50% by weight of the polylactic acid resin, (2) 25 to 75% by weight of at least one mono-fatty acid ester of a C₂-C₆ polyol such as propylene glycol, ethylene glycol, diethylene glycol, dipropylene glycol, 1,4-butane diol, pentaerythritol, trimethylolpropane, trimethylolethane, and (3) 5 to 50% by weight of at least one solvent or humectant, wherein (1), (2) and (3) together constitute at least 80%, at least 90%, or at least 95% and up to 100% of the total weight of the premix. Such a premix may contain, for example, up to 20%, up to 10%, or up to 5% by weight of a surfactant, different from (2).

In yet other specific embodiments, such a premix contains (1) 1 to 50% by weight of the polylactic acid resin, (2) 25 to 75% by weight of at least one mono-fatty acid ester of a C₂-C₆ polyol such as propylene glycol, ethylene glycol, diethylene glycol, dipropylene glycol, 1,4-butane diol, pentaerythritol, trimethylolpropane, trimethylolethane, (3) 1 to 60% by weight of at least one component 1 material, preferably at least triglyceride preferably at least triglyceride and/or at least one C₃₋₂₄ dialkyl ester of an aliphatic (preferably linear aliphatic having up to 12 carbon atoms) dicarboxylic acid (and especially at least one plant oil or animal fat), and (4) 1 to 50% by weight of at least one solvent or humectant, wherein (1), (2), and (3) together constitute at least 80%, at least 90% or at least 95% and up to 100% of the total weight of the premix. Such a premix may contain, for example, up to 20%, up to 10%, or up to 5% by weight of a surfactant, different from (2).

A representative sunscreen formulation of the invention may be a mixture that comprises the following ingredients. Additional components may be present.

Representative Sunscreen Formulation

| | | Amount |
|---|---|---|
| | | (% by |
| Ingredient | Function | Weight) |
| Water | Carrier | 30 to 70 |
| 1,3-propanol diol or | Humectant | 5 to 20 |
| other humectant | | |
| Xanthan Gum and/or | Water-soluble polymer, | 0.1 to 1 |
| Guar Gum | thickener | |
| Ensulizole | Water-soluble UV absorber | 1 to 5 |
| Phenylpropanol/caprylyl | Water-soluble | 0.25 to 2 |
| glycol | Humectant/fragrance/ | |
| | emollient | |
| Arginine | pH adjuster | 0.1 to 2 |
| Maltodextrin | Water-soluble polymer, | 0.25 to 4 |
| | film former. | |
| Homosalate, Octisalate, | Oil-soluble UV absorbers | 5 to 25 |
| and/or Octocrylene | | |
| Carboxylic acid ester | Water-immiscible solvent, | 3 to 10 |
| such as | emollient | |
| diethylhexylnapthanate, | | |
| capric/caprylic | | |
| triglyceride, | | |
| ethylhexyl palmitate | | |
| Surfactants | Emulsifiers, zinc oxide | 0.5 to 5 |
| | wetting agent | |
| Polylactic acid Resin | Film-former | 0.5 to 5 |
| Zinc Oxide | Pigment/UV absorber | 2 to 15 |

Such a sunscreen can be made by combining the aqueous phase ingredients (water water-soluble ingredients); separately combining the ingredients of the oil phase (oil-soluble UV absorbers, water-immiscible solvent, polylactic acid resin) with the surfactants, blending the zinc oxide with either the aqueous phases or the oil phase, and then combining the oil and aqueous phases. The product is an oil-in-water emulsion in which the zinc oxide particles are suspended.

A representative foundation formulation of the invention may be a mixture of the following ingredients. Additional ingredients may be present.

Representative Foundation Formulation

| | | Amount |
|---|---|---|
| | | (% by |
| Ingredient | Function | Weight)* |
| Water | Carrier | 20 to 50 |
| 1,3-propane diol or other | Humectant | 10 to 30 |
| humectant | | |
| Water-soluble polymers | Thickener | 0.25 to 5 |
| (SMA/MA copolymer, | | |
| hydroxylpropyl starch | | |
| phosphate, guar gum and/or | | |
| xanthan gum) | | |
| Maltodextrin | Water-soluble polymer, | 0 to 5 |
| | film-former | |
| Phenylpropanol/caprylyl | Water-soluble | 0 to 2 |
| glycol | Humectant/fragrance/ | |
| | emollient | |
| Ensulizole | Water-soluble UV absorber | 0.5 to 5 |
| Inorganic pigments (such as | Color | 2 to 15 |
| TiO₂, iron oxide black, | | |
| iron oxide red, iron oxide | | |
| yellow) | | |
| Surfactants | Emulsifiers, wetting agents | 0.5 to 5 |
| Water-immiscible carriers | Water-immiscible | 5 to 35 |
| (triheptanoin, octenyl | carrier/solvent for poly | |
| succinate, cetyl alcohol, | lactic acid resin | |
| diheptyl succinate, caproloyl | | |
| Fumed Silica | Rheology modifier | 0.1 to 1 |
| Polylactic acid Resin | Film-former | 0.5 to 5 |

*Based on total formulation weight.

A representative lipstick formulation of the invention may be a mixture of the following ingredients. Additional ingredients may be present.

Representative Lipstick Formulation

| | | Amount |
|---|---|---|
| | | (% by |
| Ingredient | Function | Weight)* |
| Liquid oil such as a vegetable | Carrier | 25 to 75 |
| or synthetic mono- di- or | | |
| triglyceride (e.g., castor oil, | | |
| caprylic/carpic triglyceride, | | |
| etc.) | | |
| Wax (natural and/or synthetic) | Carrier/solidifier | 10 to 30 |
| C8-24 alkane(s) | Carrier, emollient | 0 to 50, |
| | | preferably 0.25 |
| | | to 50 or 10 to 25 |
| Inorganic pigments | Colorant | 0 to 5, preferably |
| | | 0.1 to 5 |
| Surfactants | Emulsifiers, wetting | 0 to 10, |
| | agents | preferably 0 to 5 |
| | | or 0.1 to 5 |
| Humectant (1,3-propane diol, | Humectant | 0 to 20, |
| etc.) | | preferably 0.1 to |
| | | 5 |
| Preservatives (e.g., methyl | Rheology modifier | 0 to 5, preferably |
| paraben, propylparaben) | | 0.1 to 1 |
| Polylactic acid Resin | Film-former | 0.1 to 5, |
| | | preferably 0.25 |
| | | to 5 |
| Actives (vitamins, medications) | Actives | 0 to 10, |
| | | preferably 0.1 to |
| | | 5 |

*Based on total formulation weight.

Cosmetics of the invention can be used in the same general manner as conventional cosmetics formulated for the same purpose. They can be applied in a manner adapted for their intended use.

The following examples illustrate the invention, but are not intended to limit it in any way. All parts and percentages are by weight unless otherwise indicated.

### Examples 1 and 2 and Comparative Sample A

Oil-in-water sunscreens containing organic and inorganic UV absorbers (Comparative Sample A and Examples 1 and 2) are prepared as follows:

A water-dispersible premix is prepared by combining 10 parts by weight of 1,3-propanediol, 0.2 parts of xanthan gum, 0.2 parts of guar gum, 2 parts of ensulizole, 0.5 part of L-arginine, 1 part of a phenylpropanol/caprylyl glycol/1,3-propanediol mixture (Sensiva PA 40 from Schulke, Inc., Fairfield, N.J. USA), and (for Comparative Sample A only) 2 parts of maltodextrin (a water-soluble film-former) to form a viscous, homogeneous mixture. The premix then is heated to 70° C. and added to 49.1 parts of water and homogenized to produce an aqueous phase.

An oil/surfactant premix is prepared separately by combining 7.5 parts of homosalate, 2.5 parts of octisalate, 5 parts of octocrylene, 5 parts of di(ethylhexyl)naphthanate, 2 parts of an anionic surfactant (Dracorin GOC, from Symrise AG), 3 parts of a polyoxyethylene (20) sorbitan monolaurate, and (for Examples 1 and 2 only) 2 parts of a test film former at 70° C. 20 parts of zinc oxide are combined with the oil/surfactant premix, and the resulting dispersion is combined with the aqueous phase described above and homogenized to produce a white, sunscreen formulation.

The film-former in Example 1 is a 2296 g/mol Mₙ polymer made by polymerizing a mixture of about 90% meso-lactide and 10% L-lactide onto pentaerythritol (“PLA-1”). PLA-1 has a glass transition temperature of 26° C. and contains about 94% lactic units. About 55% of the lactic units are L-lactic units.

The film-former in Example 2 is a 3614 g/mol Mₙ polymer made by polymerizing a mixture of about 90% meso-lactide and 10% L-lactide onto 1-octadecanol (“PLA-2”). PLA-2 has a glass transition temperature of 14° C. and contains about 93% lactic units. About 55% of the lactic units are L-lactic units. (See Table 2 for additional physical property data on PLA 1 and PLA 2.)

Each of the sunscreens is subjected to SPF (sun protection factor) testing according to the method set forth in 21 CFR § 201.357(i) (2011). 0.05 gram of the test sunscreen is applied to a polymethyl methacrylate plate. It is spread using a circular motion for 30 seconds, using a vertical motion for 15 seconds, and then using a horizontal motion for 15 seconds. The coated plate is placed in a dark drawer for 15 minutes prior to SPF testing.

After the initial SPF testing, the coated plates are placed in an agitated 30° C. water bath for 20 minutes. The plates are then removed and allowed to dry in air. The dry plate are then again subjected to SPF testing. Results of the SPF testing are as indicated in Table 1.

TABLE 1

SPF Test Results

| Sample | Film Former | Before Washing | After Washing |
|---|---|---|---|
| A* | Maltodextrin | 97.8 | 48.7 |
| 1 | PLA-1 | 90.0 | 66.2 |
| 2 | PLA-2 | 138.2 | 68.4 |

*Comparative

Examples 1 and 2 perform comparably or better than the comparative sample before washing. Good performance on SPF testing is indicative of film formation when the sunscreen is applied; much poorer results are obtained when good film formation does not take place. Examples 1 and 2 perform much better than the comparative sample after the plates have been washed. The better wash-off resistance again is indicative of good film formation.

### Examples 3-9

Oil-in-water sunscreen Examples 3-9 are made in the following general manner. A water-dispersible premix is made by combining 20 parts of 1,3-propane diol, 0.5 parts of a xanthan gum/guar gum mixture, 2 parts of ensulizole, 0.5 parts of L-arginine, 1.0 part of Sensive PA 40, and 2 parts of maltodextrin. The resulting mixture is combined with 47 parts of 80° C. water and maintained at 80° C. This produces an aqueous phase.

An oil/surfactant/zinc oxide phase is made by mixing at 75° C. 10 parts of a 50% dispersion of zinc oxide in a caprylic/capric triglyceride carrier, 7.5 parts of homosalate, 2.5 parts of octisalate, 5 parts of octocrylene, 5 parts of ethylhexyl palmitate, 2 parts of sorbitan oleate, 3 parts of poly(propylene glycol)60 sorbitan monolaurate, and 2 parts of a film former. The film former in each case is a polylactic acid resin made by polymerizing a mixture of about 90% meso-lactide and 10% L-lactide onto an initiator, as described in Table 2. The sunscreen is then made by adding the 75° C. oil/surfactant/zinc oxide phase into the 80° C. aqueous phase and homogenizing.

TABLE 2

| | PLA | | | | % Lactide |
|---|---|---|---|---|---|
| Example | sample | Initiator | Mₙ, g/mol | T_{g}, ° C. | Units |
| 3 | PLA-3 | Lauryl alcohol | 2907 | 16.4 | 93.6 |
| 4 | PLA-4 | 1-Octadecanol | 5256 | 25 | 94.9 |
| 5 | PLA-5 | Capryl alcohol | 5261 | 33 | 97.5 |
| 6 | PLA-6 | Capryl alcohol | 2588 | 17 | 95.0 |
| 7 | PLA-7 | Capryl alcohol | 1134 | −6.5 | 88.5 |
| 8 | PLA-8 | 1-Octadecanol | 1165 | 11 | 76.9 |
| 9 | PLA-2 | 1-Octadecanol | 3615 | 14.1 | 92.6 |

The Brookfield viscosities of each of sunscreen Examples 3-9 are determined at 23° C. using a Varispeed method with a #27 spindle. SPF is measured in each case as described before. Results are as indicated in Table 3.

TABLE 3

| Example | PLA sample | Viscosity, cP | SPF |
|---|---|---|---|
| 3 | PLA-3 | 271 | 22.3 |
| 4 | PLA-4 | 2550 | 30.9 |
| 5 | PLA-5 | 1965 | 14.4 |
| 6 | PLA-6 | 2350 | 7.9 |
| 7 | PLA-7 | 515 | 9.8 |
| 8 | PLA-8 | 2940 | 18.9 |
| 9 | PLA-2 | 9780 | 22.7 |

The SPF data in Table 3 reveals a correlation between the length of the initiator of the polylactic acid resin and SPF values. In general, longer initiator chain length corresponds to better SPF values. PLA molecular weight affects viscosity but has little effect on SPF values.

### Examples 10 and 11

Foundation Examples 10 and 11 are made in the following general manner. 40 parts 1,3-propanediol, 2 parts of a styrene maleic anhydride copolymer (MSA 1800 from Cray Valley), 6 parts of ensulizole, 16 parts titanium dioxide particles, and 2 parts of Sensive PA 40 are combined. Separately, 0.4 parts of xanthan gum, 6 parts of modified cornstarch (ICB 3000 from Tate and Lyle), and 3 parts of a sodium starch octenylsuccinate/cucurbturils mixture (Aqstar M1, Aqdot Company, Cambridge, UK) are combined. The two mixtures are then homogenized together. To the homogenized materials is added a mixture of 1.4 parts iron oxide red, 4.2 parts of iron oxide yellow, and 0.4 parts iron oxide black (available from Making Cosmetic Color) followed by further homogenization. The resulting mixture is then blended with 71.6 parts of 80° C. water. The result is a dispersion of the pigments in an aqueous phase.

Separately, 30 parts of a diacetylated monoglyceride prepared from soy feedstocks, 2.5 parts of cetyl alcohol, 1.5 part of the sodium starch octenylsuccinate/cucurbturils mixture, 4 parts of a mixture of glyceryl oleate citrate and caprylic/capric triglyceride, 1 part of fumed silica, and 4 parts of a polylactic acid resin are combined at 75° C. to produce an oil/surfactant phase. The 75° C. oil/surfactant phase is then blended into the 80° C. aqueous pigment dispersion and homogenized to produce the foundation.

The polylactic acid resins in Examples 10 and 11 are PLA-1 and PLA-2, respectively.

Examples 10 and 11 are evaluated SPF rating as before, both before and after washing. Results are as indicated in Table 4.

TABLE 4

SPF Test Results

| Sample | Film Former | Before Washing | After Washing |
|---|---|---|---|
| 10 | PLA-1 | 12.7 | 9.3 |
| 11 | PLA-2 | 10.6 | 7.4 |

Both PLA film-formers impart good wash-off resistance to the foundation formulations.

### Examples 12-18 and Comparative Sample B

Foundation compositions (Examples 12-18) are made in the following general manner. A premix is made by combining 20 parts of 1,3-propanediol, 1 part of an SMA/MA copolymer, 0.7 parts of a hydroxypropyl starch phosphate, 0.3 parts of guar gum, and 2 parts of maltodextrin. Separately, a pigment mixture containing 0.3 parts of iron oxide red, 1 part of iron oxide yellow, 0.1 part of iron oxide black, and 8 parts of titanium dioxide is formed. The pigment mixture and premix are combined until uniform, and then combined with 42.5 parts of 85° C. water. This produces an aqueous phase in which the pigments are dispersed.

An oil/surfactant phase is made by mixing 5 parts of a diheptyl succinate/capryloyl glycerin sebacic acid copolymer mixture (LexfeelN50, Inolex, Inc.), 2 parts of cetyl alcohol, 1 part of a sodium starch octenylsuccinate/cucurbturils mixture (Aqstar M1, Aqdot Company, Cambridge, UK), 10 parts of a triheptanoin/C13-16 paraffin mixture (Lexfeel from Inolex), 2.0 parts of sorbitan oleate, 2.0 parts of a polyethyleneglycol 60 sorbitan monolaurate, 0.1 part of fumed silica, and 2 parts of a film former (except in Comparative Sample B, in which the film former is omitted). The film former in each case is a polylactic acid resin as indicated in Table 5. The sunscreen is then made by combining the 85° C. aqueous phase with the 75° C. oil/surfactant phase and homogenizing.

Viscosity and SPF rating are determined on each sample in the manner described before. Transfer resistance is determined by applying a preweighed sample of the foundation onto a 2 cm×2 cm square section of a human subject's forearm using a doe foot applicator and allowed to dry for one minute. The applicator is reweighed to calculate the amount of foundation applied to the skin. A weighed 3 cm×3 cm tissue is then applied to the applied foundation and dragged 3 cm. The tissue is then re-weighed to measure the amount of foundation removed. The amount transferred is then calculated as 100%×(wt. removed÷wt. applied).

Results of the testing are as indicated in Table 5.

TABLE 5

| Example | PLA resin | Viscosity, cP | SPF rating | Transfer, % |
|---|---|---|---|---|
| 12 | PLA-3 | 514 | 4.3 | 14.9 |
| 13 | PLA-4 | 2355 | 5.8 | 17.6 |
| 14 | PLA-5 | 1058 | 4.6 | 15.7 |
| 15 | PLA-6 | 290 | 2.9 | 17.0 |
| 16 | PLA-7 | 526 | 3.0 | 21.4 |
| 17 | PLA-8 | 2655 | 3.8 | 26.3 |
| 18 | PLA-2 | 1465 | 3.9 | 21.8 |
| B* | none | 2155 | 3.4 | 43% |

*Comparative.

All of the polylactic acid resins are shown to be effective film-formers, as indicated especially by the excellent transfer resistance compared with the control (Comparative Sample B). For comparison, 43.1% of the mass transfers when an otherwise identical foundation formulation, but lacking film-former, is tested.

### Examples 19-20 and Comparative Sample C

Water-in-oil sunscreen formulations are prepared as follows: 10 parts propane diol, 1 part of caprylhydroxamic acid, 0.5 parts zinc lactate, and 0.2 parts of L-arginine are mixed at room temperature with 49.3 parts of water to form an aqueous phase. Separately, an oil/surfactant/zinc oxide phase is prepared by mixing 19 parts of a C15-C19 alkane mixture (Emogreen L15, Seppic S.A.), 1 part of Dracorin GOC, and 1 part of tri(Polyglyceryl-3/Lauryl) Hydrogenated Trilinoleate. 15 parts of zinc oxide are added to the mixture and dispersed until homogenous. The oil/surfactant/zinc oxide phase is warmed to 75° C., 3 parts of a film former (as indicated in Table 6) are added, and mixed until uniform. The aqueous phase is combined with the oil phase at 75° C. and is mixed until homogenized to produce a white lotion. Viscosity and SPF both before and after washing are measured as before. Results are as in Table 6.

TABLE 6

| | | | SPF rating | |
|---|---|---|---|---|
| Example | PLA resin | Viscosity, cP | Before Washing | After Washing |
| 19 | PLA-1 | 1350 | 9.6 | 10.5 |
| 20 | PLA-2 | 1830 | 13.3 | 12.0 |
| C* | none | 2920 | 7.7 | 8.0 |

*Comparative.

The increase in SPF rating seen with Examples 19 and 20 relative to Comparative Sample C indicates the PLA resins are effective film-formers in this formulation.

### Examples 21-22

Oil-in-water sunscreen formulations containing only an inorganic sunscreen are prepared as follows: 0.3 parts of xanthan gum, 10 parts propanediol, 1 part of a caprylhydroxamic acid/glyceryl caprylate mixture, 0.5 parts of L-arginine, and 1 part of hydrolyzed corn starch hydroxy ethyl ether are mixed with 43.2 parts of water until homogenized to form an aqueous phase. The aqueous phase is heated to 75° C. An oil/surfactant/zinc oxide phase is prepared by combining 20 parts of an isoparaffin mixture (Lexfeel WOW), 15 parts of zinc oxide, 3 parts of a film-former as indicated in Table 7, 1 part of Dracorin GOC), 3 parts of sodium behenoyl lactylate are combined at 75° C. and mixed until homogenous. The phases are mixed and homogenized. Viscosity and SPF (before and after washing) are measured as before, with results as reported in Table 7.

TABLE 7

| | | | SPF rating | |
|---|---|---|---|---|
| Example | PLA resin | Viscosity, cP | Before Washing | After Washing |
| 19 | PLA-1 | 21500 | 9.0 | 10.6 |
| 20 | PLA-2 | 5900 | 9.7 | 6.7 |

### Examples 23 and 24

Lipstick formulations are produced as follows: 30.8 parts of castor oil, 16 parts of caprylic/capric triglyceride, 17 parts of isoeicosane, 5 parts of meadowfoam seed oil, 3.5 part of microcrystalline wax, 3.5 part of ozokerite wax, 7 parts of candelilla wax, 3 parts of carnauba wax, and 1 part of a film-former as indicated in Table 8 are combined at 80-95° C. to form a molten wax phase. Separately, 1 part of Red No. 7 D&C Lake and 11 parts of mica are combined, and then mixed into the molten wax phase. 0.2 parts of tocopherol and 1 part of paraben-DU (mixture of 56% propylene glycol, 3% propylparaben, 11% methylparaben and 30% diazolidinyl urea) are mixed and then combined with the molten wax phase. The resulting lipstick formulations are poured into a lipstick mold and solidified in a freezer.

The lipsticks are tested for transfer resistance in the manner described before. Hardness and penetration force are measured according to ASTM D1321-10. Results are as indicated in Table 8.

TABLE 8

| | | Hardness (g- | Penetration Force |
|---|---|---|---|
| Sample | % Transfer | force) | for 7 mm |
| PLA-1 | 12 | 442 | 131 |
| PLA-2 | 13 | 380 | 126 |

### Examples 24-33 and Comparative Sample A

Comparative Sample A: 20 parts of coconut oil and 3 parts of PLA-1 are heated separately to 90° C. and mixed by hand. The resulting mixture is homogeneous upon visual inspection at that temperature. When this experiment is repeated at 80° C. the mixture is only partially homogeneous and is entirely heterogeneous when the mixture is made at 70° C. When the coconut oil and PLA-1 are mixed at a 3:1 ratio at 80° C., the resulting mixture is completely heterogenous.

Example 24: Glycerol monostearate and coconut oil are combined at a 68/32 weight ratio. Entirely homogeneous mixtures are obtained when 20 grams of that mixture and 3 parts of PLA-1 are separately heated to 70° C. and combined. Similar results are obtained when the mix ratio is 3:1. Upon cooling, these mixtures partially phase separate but are easily combined with an aqueous phase at 70° C. to produce a cosmetic formulation.

Example 25: Glycerol monooleate and coconut oil are combined at a 84/16 ratio. Partially heterogeneous mixtures are obtained when 20 grams of that mixture and 3 parts of PLA-1 are separately heated to 70° C. and combined; at 80° C. the mixtures are entirely homogeneous. When the mix ratio is 3:1, partially heterogeneous mixtures are obtained at both the 70° C. and 80° C. mixing temperatures. Upon cooling, these mixtures partially phase separate but are easily combined with an aqueous phase at 70° C. to 80° C. to produce a cosmetic formulation.

Example 26: Glycerol monooleate and glycerol monolaurate are combined at a 63/37 ratio. Partially heterogeneous mixtures are obtained when 20 grams of that mixture and 3 parts of PLA-1 are separately heated to 70° C. and combined; at 80° C. the mixtures are entirely homogeneous. When the mix ratio is 3:1, completely homogeneous mixtures are obtained at both the 70° C. and 80° C. mixing temperatures. Upon cooling, these mixtures partially phase separate but are easily combined with an aqueous phase at 70° C. to 80° C. to produce a cosmetic formulation.

Example 27: Propylene glycol monooleate and coconut oil are combined at a 99/1 ratio. Fully homogeneous mixtures are obtained when 20 grams of that mixture and 3 parts of PLA-1 are separately heated to 80° C. and combined. Upon cooling, these mixtures partially phase separate but are easily combined with an aqueous phase at 80° C. to produce a cosmetic formulation.

Example 28: Glycerol monostearate and coconut oil are combined at a 63/37 ratio. Partially heterogeneous mixtures are obtained when 20 grams of that mixture and 3 parts of PLA-1 are separately heated to 80° C. and combined. Upon cooling, these mixtures partially phase separate but are easily combined with an aqueous phase at 70° C. to 80° C. to produce a cosmetic formulation.

Example 29: Propylene glycol monooleate and coconut oil are combined at a 55/45 ratio. Partially heterogeneous mixtures are obtained when 20 grams of that mixture and 3 parts of PLA-1 are separately heated to 80° C. and combined. Upon cooling, these mixtures partially phase separate but are easily combined with an aqueous phase at 80° C. to produce a cosmetic formulation.

Example 30: Glycerol monostearate and coconut oil are combined at a 68/32 ratio. Partially heterogeneous mixtures are obtained when 20 grams of that mixture and 3 parts of PLA-4 are separately heated to 80° C. and combined. When the mix ratio is 3:1, partially heterogenous mixtures are obtain at both 70° C. and 80° C. Upon cooling, these mixtures partially phase separate but are easily combined with an aqueous phase at 70° C.-80° C. to produce a cosmetic formulation.

Example 31: Glycerol monooleate and coconut oil are combined at a 84/16 ratio. Partially heterogeneous mixtures are obtained when 20 grams of that mixture and 3 parts of PLA-4 are separately heated to 70° C. and combined; at 80° C. the mixtures are entirely homogeneous. Upon cooling, these mixtures partially phase separate but are easily combined with an aqueous phase at 70° C. to 80° C. to produce a cosmetic formulation.

Example 32: Glycerol monooleate and glycerol monolaurate are combined at a 63/37 ratio. Partially heterogeneous mixtures are obtained when 20 grams of that mixture and 3 parts of PLA-4 are separately heated to 80° C. Similar results are obtained when the mix ratio is 3:1. Upon cooling, these mixtures partially phase separate but are easily combined with an aqueous phase at 80° C. to produce a cosmetic formulation.

Example 33: Propylene glycol monooleate and coconut oil are combined at a 99/1 ratio. Fully homogeneous mixtures are obtained when 20 grams of that mixture and 3 parts of PLA-4 are separately heated to 80° C. and combined. Upon cooling, these mixtures partially phase separate but are easily combined with an aqueous phase at 80° C. to produce a cosmetic formulation.

### Examples 34 and 35

Example 34: 6.0 parts of coconut oil, 10.2 parts of glycerol monostearate and 5.4 parts of PLA-4 are combined, heated to 80° C. for one hour, and cooled to room temperature to form a premix.

Separately, a water phase is prepared by mixing 44.55 parts water, 0.55 parts xanthan gum and 9.9 parts of 1,3-propanediol.

A lotion is prepared by heating the premix to 80° C. and combining it with 18 parts of coconut oil, 1.8 parts of glyceryl oleate citrate and 3.6 parts of steareth-21 surfactant to produce an oil phase. The water phase is heated separately to the same temperature. The oil phase is poured slowly into the water phase under mild agitation conditions of about 400 rpm. The agitation rate is increased to 1000 rpm and is continued for 5 minutes after the phases are combined. The resulting product is a homogeneous lotion that is resistant to phase separation.

Example 35 is made in the same manner, except PLA-4 is replaced with PLA-1. Similar results are obtained.

1. A cosmetic composition for applying a film on lips, skin, or eyelashes, the cosmetic composition comprising (component 1) at least one of (i) a water-immiscible organic compound having a molecular weight of up to 1000 g/mol, a melting temperature no greater than 60° C., and a boiling temperature at 101 kPa pressure of at least 100° C., and (ii) a water-immiscible silicone compound; and (2) an amorphous grade polylactic acid resin containing at least 70% by weight of lactic repeating units and having a glass transition temperature of no greater than 65° C., wherein the polylactic acid resin has a number-average molecular weight of 1000 to 12,000 g/mol and contains a residue after removal of hydroxyl groups of a hydroxyl-containing initiator compound having 2 to 24 carbon atoms and 1 to 8 hydroxyl groups, the polylactic acid resin constituting 0.1 to 10% of the total weight of the cosmetic composition and being dissolved in component 1 or an oil phase in which component 1 also is dissolved.

2. (canceled)

2. The cosmetic composition of claim 1 wherein the polylactic acid resin is a polymer of meso-lactide or a copolymer of a lactide mixture containing at least 75% meso-lactide and at most 25% of another lactide.

3. The cosmetic composition of claim 2 wherein the polylactic acid resin furthers contain repeating units of at least one other monomer that is copolymerizable with lactide or lactic acid.

4. The cosmetic composition of claim 1 wherein the polylactic acid resin is represented by the structure: 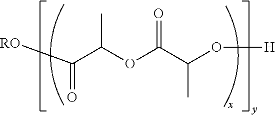
wherein y is 1 to 8, x is a positive number such that the polylactic acid resin has a number-average molecular weight of 1000 to 12,000 g/mol and R is the residue after removal of hydroxyl groups of a hydroxyl-containing initiator compound having y hydroxyl groups.

5. The cosmetic composition of claim 1 wherein the polylactic acid resin has a number average molecular weight of 2000 to 8000 g/mol.

6. The cosmetic composition of claim 1 wherein the hydroxyl-containing initiator compound is a linear or branched aliphatic monoalcohol having 6 to 20 carbon atoms.

7. The cosmetic composition of claim 1 wherein the hydroxyl-containing initiator compound is a linear or branched aliphatic alcohol having 3 to 9 carbon atoms and 2 to 4 hydroxyl groups.

8. The cosmetic composition of claim 1 wherein component 1 includes one or more of a fatty acid ester, a fatty alcohol, or an organic sunscreen.

9. The cosmetic composition of claim 1 which is an oil-in-water or water-in-oil emulsion.

10. The cosmetic composition of claim 9 that contains 5% to 90% by weight water.

11. The cosmetic composition of claim 10 which further comprises at least one water-soluble polymer.

12. The cosmetic composition of claim 1 which further comprises at least one emulsifier.

13. The cosmetic composition of claim 1 which further contains at least one particulate pigment.

14. The cosmetic composition of claim 1 which further comprises at least one of zinc oxide and titanium dioxide.

15. The cosmetic composition of claim 1 which further comprises at least one polyalcohol having 3 to 6 carbon atoms and a molecular weight of up to 125 g/mol.

16. The cosmetic composition of claim 1 which contains at least one organic UV absorber.

17. The cosmetic composition of claim 1 which is a sunscreen having a sun protection factor of at least 5 as measured according to the method described in 21 CFR § 201.357(i) (2011).

18. The cosmetic composition of claim 1 which is a sunscreen having a sun protection factor of at least 20 as measured according to the method described in 21 CFR § 201.357(i) (2011).

19. The cosmetic composition of claim 1 wherein the polylactic acid resin contains 20% to 80% of one lactic acid enantiomer and correspondingly 80% to 20% of the other lactic acid enantiomer, based on the total weight of lactic units in the polylactic acid resin.
